(19) United States
(12) Patent Application Publication
(10) Pub. No.: US 2014/0039266 A1
(43) Pub. Date: Feb. 6, 2014

(54) Disposable Speculum with Smoke Evacuator

(71) Applicant: Gadi Porat, Jerusalem (IL)

(72) Inventor: Gadi Porat, Jerusalem (IL)

(73) Assignee: ASSOCIATION FOR PUBLIC HEALTH, Jerusalem (IL)

(21) Appl. No.: 14/112,242

(22) PCT Filed: Jan. 26, 2012
(86) PCT No.: PCT/IL12/00046

§ 371 Date: Oct. 17, 2013

Related U.S. Application Data
(60) Provisional application No. 61/631,077, filed on Dec. 27, 2011.
(60) Provisional application No. 61/457,522, filed on Apr. 18, 2011.

(51) Int. Cl. A61B 1/32; A61B 1/012; A61B 1/06; A61B 1/00; A61B 1/303

(52) U.S. Cl. CPC A61B 1/32; A61B 1/00094; A61B 1/303; A61B 1/06; A61B 1/012; A61B 1/00103
USPC 600/205

(57) ABSTRACT

A disposable plastic vaginal speculum constructed for heavy duty use and/or for long duration gynecological procedures including vaginal surgical and electrosurgical procedures. The hinge region of the speculum is reinforced so that the speculum can bear forces of a magnitude normally bearable only by metal vaginal specula. The reinforcing element also functions as a smoke evacuation element.

### FIELD OF THE INVENTION

The present invention relates to the field of disposable vaginal specula.

### BACKGROUND OF THE INVENTION

Disposable, plastic vaginal specula are known. They are often formed of a moldable synthetic resin, such as polystyrene or polypropylene, and can be transparent or opaque, clear or colored.

Disposable vaginal specula are particularly useful for high-volume users, such as clinics which specialize in the detection of venereal disease or cervical cancer. Because of the large number of women being examined, it is advantageous to have a low-cost, disposable instrument obviating the need for cleaning and sterilization of the instrument after use. This is particularly true with the increase in occurrence of sexually transmitted diseases such as AIDS.

Gynecologists prefer plastic specula as they are more user-friendly, not being cold to patients as metal specula are. Additionally, metal specula have to be polymer coated for thermal and electric insulation when used for electrosurgical procedures. Any uncoated metal speculum may lead to serious accidents causing burns when such procedures are performed. Similarly, any damage to the polymer coat may result in serious clinical harm to the patient. Therefore, plastic specula are generally preferred by physicians, but such specula must be sufficiently strong and unbreakable during use, as discussed herein below.

Because the practitioner or clinician should have both hands free during an examination, a vaginal speculum is preferably lockable at any of several open positions, and then easily released for removal. However, there are problems in constructing plastic specula which can reliably remain in an open, locked position. When pressure is exerted on the speculum's blades by the vaginal wall, the locking parts or blades of the speculum can distort. It is known that plastic specula do not possess the required mechanical strength for all medical procedures. Heavy duty procedures such as in vitro fertilization (IVF) and procedures on obese patients are generally not performed using plastic specula because of their tendency to fracture at loads greater than about 2 to 3 kg. The fractured plastic generates sharp pieces with a concomitant risk of injury to the patient.

Another problem of prior-art disposable vaginal specula is that they often produce noisy clicks when being opened within the patient. This noise is caused by the ratchet action of the speculum's locking mechanism. Although the ratchet action itself is not harmful, the clicking noise is often disturbing to the patient. It sometimes causes an involuntary response on the part of the patient, often leading to sudden increases in pressure on the speculum causing it to break.

Typical plastic vaginal specula are disclosed, for example, in U.S. Pat. Nos. 3,568,665; 3,752,149; 3,246,646; 3,332,414; 3,650,266; 3,985,125, 3,890,961 and US Publ Pat Appl. 2009/177044.

Various gynecological surgical procedures have recently been developed based on electrosurgical and laser technologies. Among these surgical techniques are those involving gynecological procedures such as, for example, hysterectomies and surgical treatment of cervical cancers. During these types of gynecological procedures, a vaginal speculum is typically employed to dilate the vaginal cavity so that the uterus or cervix may be operated upon in an unobstructed manner. Electrosurgical tools are then inserted through the speculum and the surgical procedures are carried out.

Electrosurgical techniques often involve vaporization of tissue which necessarily produces smoke. This smoke can obscure the surgeon's view of the area undergoing surgery. In the area of gynecological surgery, vaginal specula including tubes for removing the smoke produced by electrosurgical procedures have been suggested. One such device is disclosed in U.S. Pat. No. 4,884,559 to Collins. The Collins speculum includes a lower blade and an upper blade and is provided with a separately mounted smoke tube or an internally formed hollow, each of which extends over substantially the entire length of the upper blade. The smoke evacuating tube is attached to the concave portion of the upper blade using inserts.

Similarly, U.S. Pat. No. 5,499,964 to Beck teaches a U-shaped channel member positioned adjacent to the contoured inner surface of the upper blade member of a speculum. The U-shape channel member is configured to snap fit into the upper blade forming a channel through which the smoke produced by the gynecological procedure is withdrawn.

U.S. Pat. No. 2,483,233 to Price et al teaches a speculum with an air tube that is welded to the speculum and is used as an air path for evacuating smoke from a gynecological electrosurgical procedure.

Prior art vaginal specula incorporating smoke evacuation systems have employed smoke tubes that are either welded or soldered to metal specula, solvent bonded to plastic specula blades, or in the case of the Collins device, require additional fasteners spaced along the upper blade. These tube systems have limited rates of evacuation due to relatively small tube diameters and require fastening means which involves added assembly time and/or manufacturing expense.

In view of the above remarks it would be advantageous to develop a sturdy plastic vaginal speculum which would not fracture or collapse under loads normally encountered during examinations of long duration or during extended gynecological surgical procedures. Additionally, a robust plastic speculum equipped with a low cost, easily manufactured and applied smoke evacuator member would be desirable. Most desirable would be a speculum solving both such problems simultaneously.

### SUMMARY OF THE PRESENT INVENTION

It is an object of the present invention to provide a sturdy disposable vaginal speculum that allows for the performance of long-duration, heavy duty medical procedures.

It is a further object of the present invention to provide a safe, substantially non-breakable, disposable speculum that can withstand loads in excess of about 2 to 3 kg, preferably in excess of 6 kgs.

It is yet a further object of the present invention to rapidly evacuate smoke from a site undergoing electrosurgical gynecological procedures so that the surgeon's view of the site is not obscured during the procedures.

An additional object of the present invention is to evacuate smoke through a vaginal speculum including an illumination system for illuminating an area undergoing surgery so that light illuminating the area under surgery is not obscured by the smoke.

These and other objects of the present invention may be readily understood from the discussion below and the Figures shown herein.

In one aspect of the present invention, there is provided a vaginal speculum which includes a plastic dorsal member, a plastic ventral member and an insert support element. The plastic dorsal member has a distal end and a proximal end and includes: i. a rounded dorsal blade member with concave and convex surfaces; ii. a dorsal handle, and iii. a pair of lateral wall elements connecting between, and formed integrally with, the blade member and the handle so as to form an inspection aperture between the concave surface of the blade member and an opposing portion of the handle. Each wall element has formed therewith a first hinge portion. The plastic ventral member includes: i. a rounded ventral blade member; and ii. a ventral handle having a portion adapted to fit between the lateral wall elements of the dorsal member and having formed thereon a pair of second hinge portions adapted for interconnection with the first hinge portions. The first and second hinge portions form a hinge between the dorsal member and the ventral member for facilitating relative angular translation between these members. The insert support element is in mechanical connection with the proximal end of the dorsal blade member, arranged within the inspection aperture, and configured to extend within the concave surface of the dorsal blade member so as to cooperate therewith to form a channel for the evacuation of smoke or detritus produced by a gynecological procedure when suction from a suction source is applied. The support element is also in load-bearing arrangement with a predetermined one of the first or second pairs of hinge portions, thereby reducing the likelihood of shear stress induced mechanical failure of the dorsal member when in use.

Relative angular rotation of the speculum is effected about an axis extending transversely through the hinge, adapted to support thereat shear force components normal to the axis above the hinge.

The insert support element includes: i. a seat having a proximal portion formed to fit within the concave surface of the dorsal blade element, the seat having a distal portion extending generally away from the inspection aperture; and ii. a pair of lateral fenders extending generally transversely away from the seat along the pair of lateral wall elements so as to be in load-bearing arrangement with the hinge. This reduces the likelihood of shear stress induced mechanical failure of the speculum when in use. Forces acting on the hinge are partially transferred by the fender elements to the arched proximal end of the dorsal blade member.

In some embodiments of the vaginal speculum, the insert support element further includes a hollow nipple adapted for connection to a suction source to evacuate smoke and/or detritus produced as a result of the gynecological procedure.

In yet other embodiments of the speculum, the insert support element further includes a retainer ring through which an electrical connection of a light source or an optical instrument may be positioned.

In further embodiments of the speculum, the speculum is formed of a high stress resistant plastic suitable for supporting stresses in excess of at least a force of 6 kgs without bending and without fracturing.

In another embodiment of the speculum, each of the first hinge portions includes a hinge pin and each of the second hinge portions includes a hinge hole. In yet other embodiments, each of the second hinge portions includes a hinge pin and each of the first hinge portions includes a hinge hole.

In yet another embodiment of the speculum, each of the lateral wall elements of the dorsal member includes a hinge pin lead slot leading to the hinge holes from the distal edge of the lateral wall elements along their inside surfaces.

In a further embodiment of the vaginal speculum, the dorsal blade member and the dorsal handle member are integrally joined and form an arcuate region at the proximal end of the dorsal blade member.

In another embodiment of the speculum, the forces acting on the hinge portions are transferred by the fender elements to the arcuate region of the seat, the arcuate region of the seat, at least partially supporting the arcuate region at the proximal end of the dorsal blade member.

In another embodiment of the speculum, shear forces acting on the hinge portions are dispersed therefrom because of the greater area of the speculum upon which they act above the hinge region than below it. This greater area is a result of the fender elements being positioned essentially above the hinge region.

In still another embodiment of the vaginal speculum of the invention, the second hinge portions are hinge pins and the first hinge portions are elongated grooves fitted with a ratchet mechanism into which the hinge pins are inserted.

In still other embodiments of the vaginal speculum, the speculum contains a locking mechanism in mechanical communication with the dorsal and ventral handle members operable to lock the dorsal blade member and the ventral blade member in a user-selected position with respect to each other.

In some embodiments of the speculum, the locking mechanism includes an at least partially threaded screw that is joined to and extends from the dorsal handle member through the ventral handle member. The screw has a nut positioned on it for holding the ventral and dorsal blade members in a predetermined user-selected position with respect to each other.

In other embodiments of the speculum, the locking mechanism is a trigger mechanism in mechanical communication with the dorsal and ventral handles. The trigger mechanism is operable to lock the handles so that the dorsal and ventral blade members remain in a user selected position with respect to each other. The trigger mechanism includes a trigger element configured so that when pressed in a first direction the trigger element is operative to lock the ventral and dorsal handles, preventing rotation and/or translation of the blade members. When pressed in a second direction the trigger element is operative to unlock the handles, allowing rotation and/or translation of the blade members. In some embodiments of the trigger mechanism, the mechanism includes: a pair of substantially parallel positioning projections, the projections separated from each other by a predetermined distance and projecting generally transversely from the dorsal handle in a distal direction; and a trigger element having a proximal press end and a distal wedge end, the wedge end being formed to be thicker than the remaining sections of the trigger element. When the press end is pressed in a first direction the wedge end is moved between the positioning projections and wedged between the projections, thereby locking the ventral and dorsal handles, preventing rotation and/or translation of the blade members. When the press end is pressed in a second direction the wedge end moves out of, and away from, the positioning projections unlocking the ventral and dorsal handles, allowing rotation and/or translation of the blade members.

In another aspect of the present invention, there is provided an insert support element for use with a vaginal speculum, the speculum configured to include an inspection aperture, hinge pins forming part of a hinge and a dorsal blade element having a concave surface. The insert support element includes: i. a seat having a proximal portion formed to fit within the concave surface of the dorsal blade member and having a distal portion extending generally away from the inspection aperture; and ii. a pair of lateral fenders extending generally transversely away from the seat so as to be in load-bearing arrangement with the hinge pins of the speculum. This reduces the likelihood of shear stress induced mechanical failure of the speculum when in use. The distal portion and the proximal portion of the seat are configured so as to cooperate with the concave surface of the dorsal blade member of the speculum to form a channel for the evacuation of smoke or detritus produced by a gynecological procedure when suction from a suction source is applied.

### DEFINITIONS

Ventral member—In the specification below this member of the speculum may also be described as the fixed member or the lower member.

Dorsal member—In the specification below, this member of the speculum may also be described as the movable member or the upper member.

Proximal—the direction closest to the user of the speculum.

Distal—the direction furthest from the user of the speculum

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only. They are presented in the cause of providing what is believed to be the most useful and readily understood description of the principles and conceptual aspects of the invention. In this regard, no attempt is made to show structural details of the invention in greater detail than is necessary for a fundamental understanding of the invention. The description taken with the drawings make apparent to those skilled in the art how the several forms of the invention may be embodied in practice.

In the drawings:

FIG. 1A is an isometric view of a disposable speculum constructed according to an embodiment of the present invention, the speculum being in its open configuration;

FIG. 1B is a side view of a disposable speculum constructed according to the embodiment in FIG. 1A, the speculum being in its closed configuration;

FIG. 2 is a front view of the blade profile of the disposable speculum of FIGS. 1A and 1B, the speculum being in its closed configuration;

FIG. 3 is an isometric view of the ventral member of the speculum shown in FIGS. 1A and 1B;

FIG. 4A is an isometric view of the insert support element of the speculum shown in FIGS. 1A and 1B;

FIG. 4B is a side schematic view of the insert support element of the speculum shown in FIGS. 1A and 1B;

FIG. 5 is an isometric view of the insert support element shown in FIGS. 4A and 4B outfitted with a light source;

FIG. 6A is a view of the forces acting on the hinge region of the speculum shown in FIGS. 1A and 1B and their transfer therefrom;

FIG. 6B is a view of the speculum in FIGS. 1A and 1B, connected to a suction source, the Figure showing the axes used in FIG. 6C;

FIG. 6C shows cross-sectional views along axes AA′ and BB′ of FIG. 6;

FIG. 7 is a side view of a speculum in its open configuration constructed according to another embodiment of the present invention;

FIGS. 8A-8C are side, isometric and proximal views, respectively, of an insert support element usable with the speculum in FIG. 7;

FIG. 8D shows the relationship between the dorsal blade of the speculum in FIG. 7 and the insert support element of FIGS. 8A-8C;

FIGS. 9A-9B are side and isometric views, respectively, of a lock element used with the speculum shown in FIG. 7;

FIGS. 10A-10B present the lock element of FIGS. 9A and 9B as it is positioned in the speculum of FIG. 7;

FIGS. 11A-11B show the lock element of FIGS. 9A-9B in its unlocked and locked position, respectively; and

FIG. 12 is a proximal view of the speculum shown in FIG. 7.

Similar elements in the Figures are numbered with similar reference numerals.

### DESCRIPTION OF PREFERRED EMBODIMENTS

Currently available disposable plastic vaginal specula are structurally too weak to be employed for anything other than routine short-duration examinations, including examinations where vaginal or cervical smears are obtained. In heavy duty and/or long-duration gynecological surgical procedures, a patient may reflexively or otherwise generate sudden large mechanical loads on the plastic device resulting in its fracturing. Additionally, if the plastic vaginal speculum must be held open for relatively long durations, blade bending and even collapse may occur. Changes are therefore required in the construction of plastic specula so that they may tolerate actual encountered loads.

Currently available plastic specula may support loads of up to about two to three kilograms. Since structural failure is so common with plastic specula, many, if not most, plastic specula designs have been withdrawn from the market incurring the wrath of regulatory agencies such as the FDA.

The present invention teaches a disposable plastic speculum that includes an insert support element that also provides for easy withdrawal of smoke generated by electrosurgical and laser ablation gynecological procedures. The physician then may easily proceed with his/her gynecological examination without obstruction by smoke and detritus. The insert support element also provides additional structural strength to the plastic speculum allowing such specula to support loads in excess of about six kilograms over sustained periods. These loads are similar to those supported by metal specula.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

It is to be appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

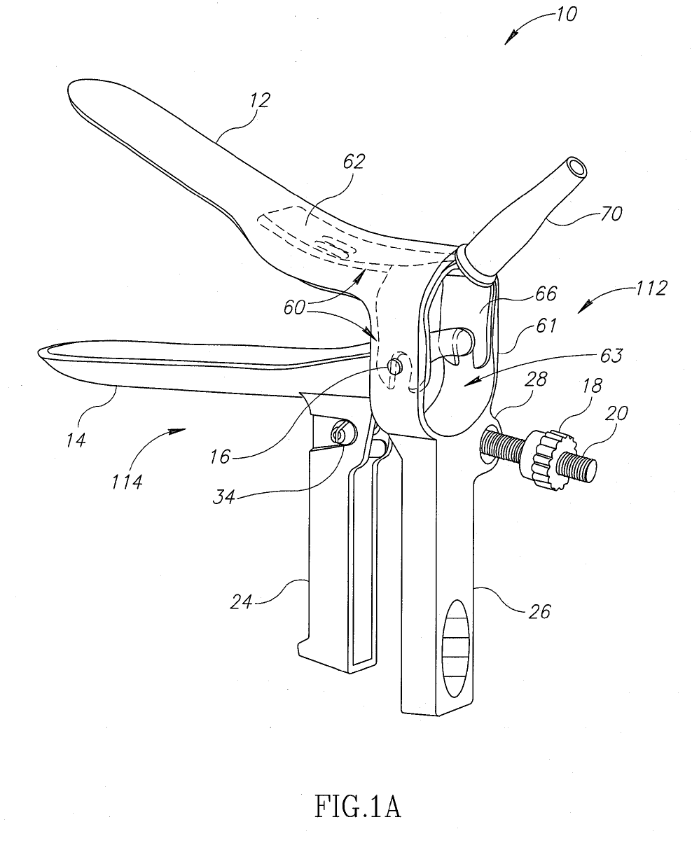

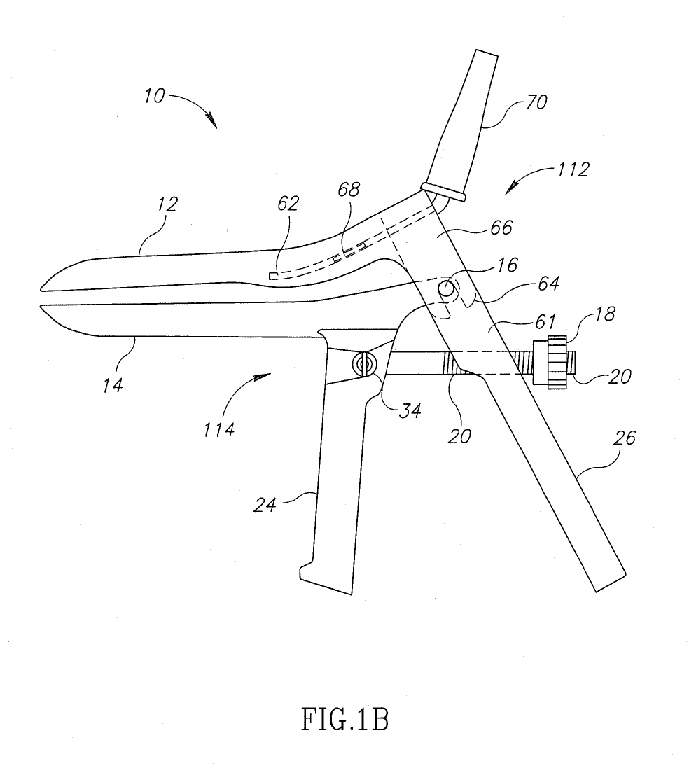

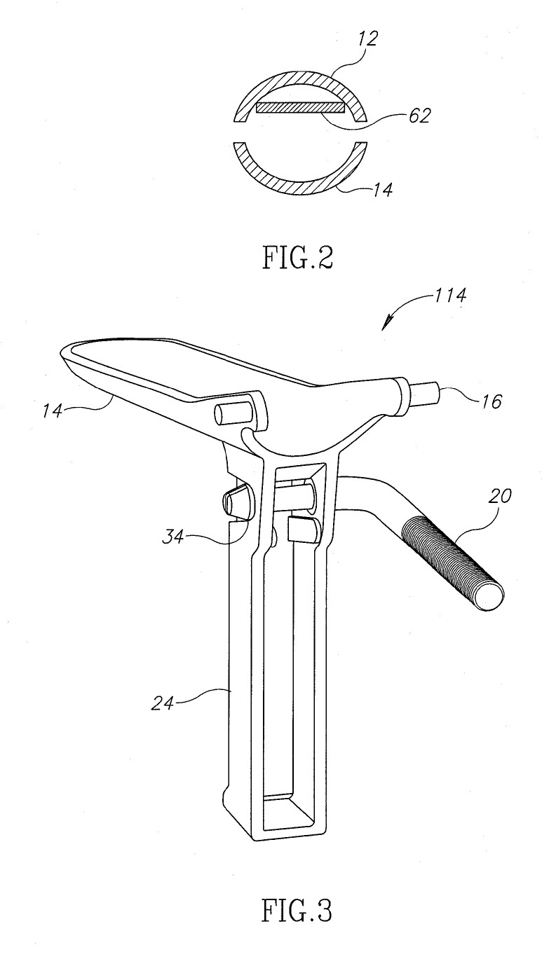

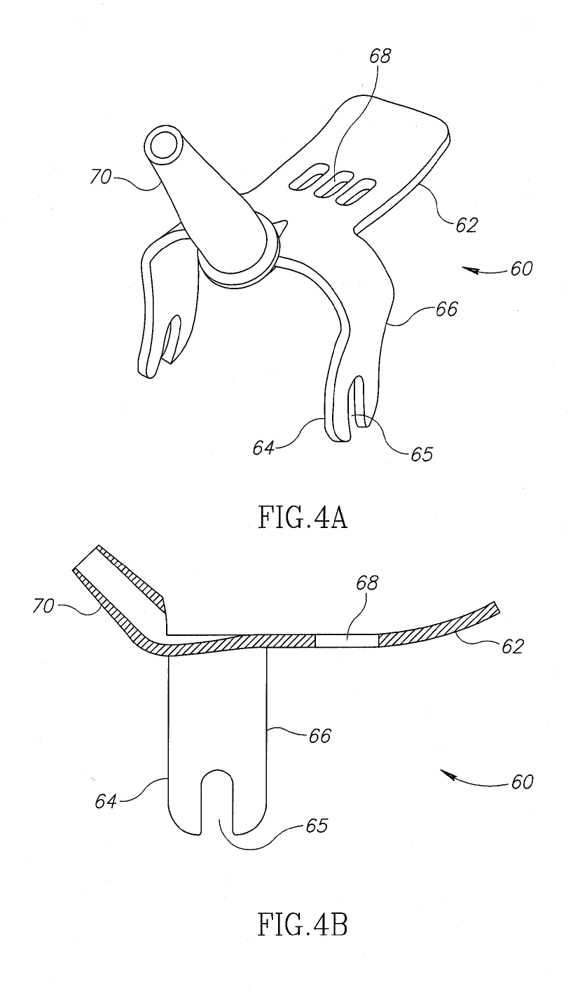

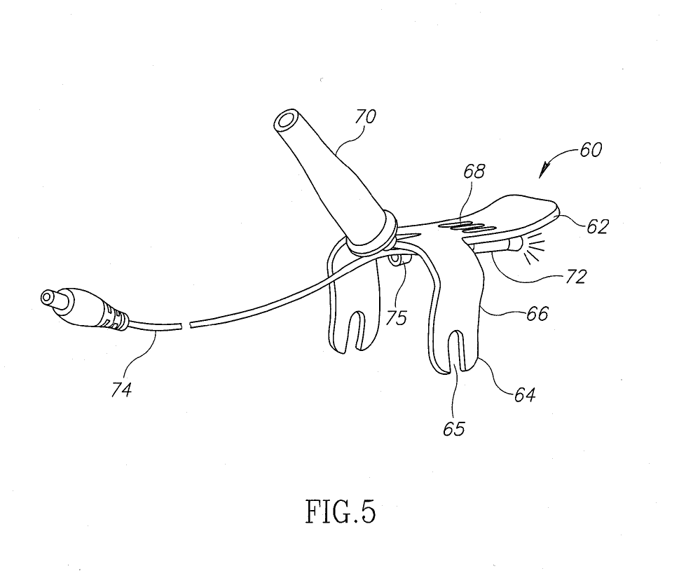

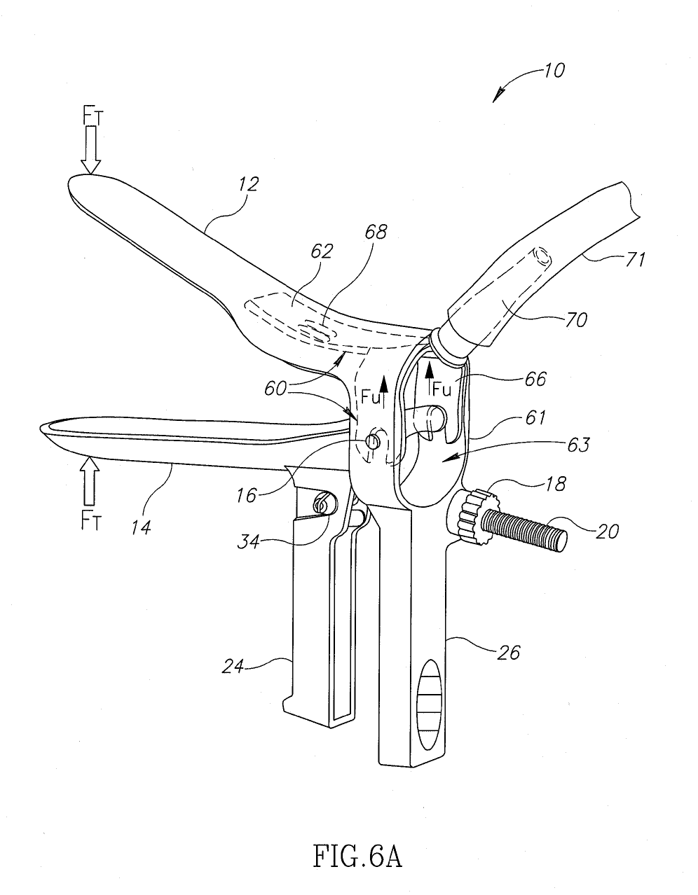

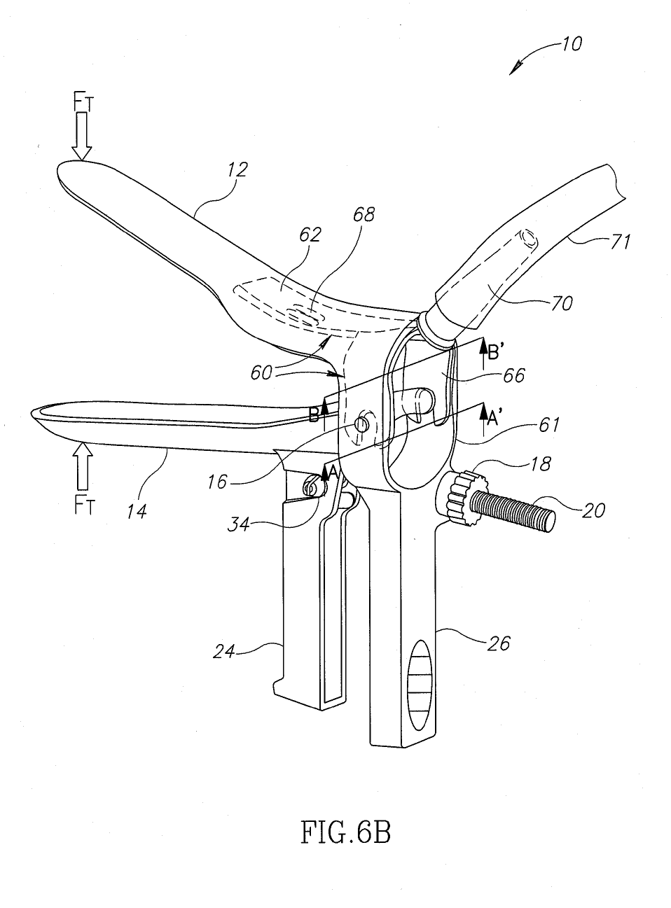

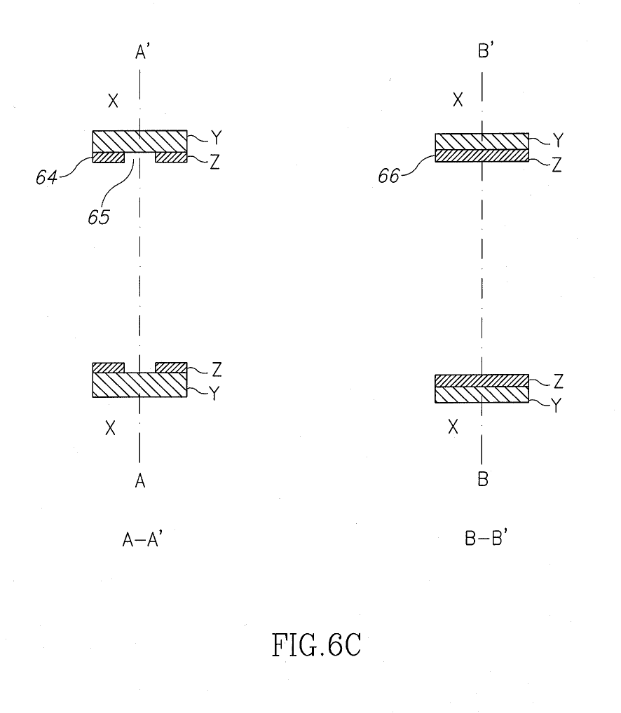

Reference is now made to FIGS. 1A-6C in which an embodiment of a disposable speculum 10 and a novel insert support element 60 is shown. FIGS. 1A-1B are isometric and side views, respectively, of speculum 10 in its open and closed configurations, respectively; FIG. 2 is a front view of the blade profile of disposable speculum 10 in its closed configuration; FIG. 3 shows an isometric view of the ventral member of the speculum in FIGS. 1A and 1B; FIGS. 4A and 4B are isometric and side views, respectively, of an insert support element of speculum 10 in FIGS. 1A and 1B; FIG. 5 is an isometric view of the insert support element of speculum 10 shown in FIGS. 1A and 1B outfitted with a light source; and FIGS. 6A-6C show how the forces acting on speculum 10 may be dispersed when speculum 10 is used.

Reference is now made to FIGS. 1A and 1B. Speculum 10 shown therein is formed of an injection molded plastic, in the present invention a high strength plastic, such as polycarbonate. Other resins possessing similar mechanical strength my also be used to form the specula of the present invention. In all events, the plastic must be stronger and more durable than the currently employed polystyrene or polypropylene. Typically, the plastic is a clear transparent plastic but colored or non transparent high strength plastics may also be used.

Speculum 10 is formed of a dorsal member 112 and a ventral member 114. Dorsal member 112 contains: a dorsal blade member 12; a dorsal handle 26 integrally formed with blade member 12; a pair of hinge pin holes (not shown) one on each lateral wall 61 of dorsal member 112 and each positioned to receive a hinge pin 16 (discussed below); and a dorsal member bolt hole 28 typically formed as an integral extension from dorsal handle 26. In other embodiments, bolt hole 28 may be formed directly in handle 26 without need of a structural extension.

Ventral member 114 includes: a ventral blade member 14; a ventral handle 24 integrally formed with blade member 14; and integrally formed hinge pins 16, one on each side of ventral member 114 near its proximal end. Hinge pins 16 can also be formed as studs, trunnions and the like. Hinge pins 16 are insertable into the hinge pin holes (not shown) on dorsal member 112 described above, thereby engaging dorsal and ventral members 112 and 114 so that speculum 10 is ready for use. Hinge pins 16 and hinge pin holes (not shown) form a hinge around which dorsal blade member 12 pivots when speculum 10 is brought from its closed to its open configuration or vice-versa. In the closed configuration, dorsal blade member 12 lies facing and substantially adjacent to ventral blade member 14 as shown in FIG. 1B.

In FIGS. 1A and 1B, insert support element 60, discussed in greater detail below in conjunction with FIGS. 4A and 4B, is shown with broken lines.

In FIG. 2, to which reference is now made, a front profile of blade members 12 and 14 of speculum 10 discussed in conjunction with FIGS. 1A, 1B and 3 below is shown. The blade members are in their closed configuration. Seat 62 of insert support element 60 also discussed below is shown. Seat 62 and the concave side of dorsal blade member 12 form a channel through which smoke and other detritus may be evacuated from speculum 10.

Returning once again to FIGS. 1A and 1B, in some embodiments there is a hinge pin lead notch (not shown) integrally formed on each of the inside faces of lateral wall elements 61 of dorsal member 112. The inside surface of each of lateral wall elements 61 is the surface that partially defines inspection aperture 63. The notch typically runs from a hinge pin hole (not shown) to a point on the distal edge of lateral wall elements 61 of dorsal member 112. The notch is intended to allow for easier positioning of hinge pins 16 in their respective hinge pin holes by sliding pin 16 along the notch until it enters its corresponding hinge hole. Without intending to limit the invention, the hinge pin lead notches may be made by using the side core method of injection molding.

An at least partially threaded bolt or screw 20 extends into and through dorsal member bolt hole 28 and then into at least one ventral member bolt hole 34 (better seen in FIG. 3) in ventral handle 24. In FIG. 1A, bolt or screw 20 makes an approximately right angle turn after extending from dorsal member bolt hole 28, entering ventral member bolt holes 34 on ventral handle 24. Without intending to be limiting, the bolt or screw is typically made of a plastic, such as nylon, polyethylene, polypropylene, acetal (polyoxymethylene) and polycarbonate. The bolt or screw may have a splayed end as best seen in FIGS. 1A and 1B which after passing through ventral member bolt holes 34 expands to engage and hold the bolt or screw to ventral handle 24. Other methods of engagement known to those skilled in the art may also be used. In other embodiments, bolt or screw 20 may pass directly from dorsal member bolt hole 28 to a single ventral member bolt hole 34 without requiring a right angle turn. In such an embodiment, holes 28 and 34 may be integrally formed directly within dorsal handle 26 and vertical handle 24, respectively, without need for any extensions from the handle members. Such a lock mechanism may be denoted as a key lock mechanism.

Plastic nut 18 sits on the side of dorsal member bolt hole 28 distal from ventral handle 24. After speculum 10 is brought from its closed configuration substantially as in FIG. 1B to its partially or completely open configuration as in FIG. 1A, nut 18 is used to lock the handles in the position desired by the physician while he carries out the required gynecological procedures.

Nut 18 and threaded bolt or screw 20 together form a locking mechanism and may be described herein as such. It should be evident to one skilled in the art that a nut and bolt mechanism is not the only locking mechanism that can be used and therefore its use here should not be deemed to be limiting.

While what has been described herein has described a locking mechanism employing a threaded bolt or screw 20 extending from dorsal handle 26 to the ventral handle 24, other locking mechanisms readily known to those skilled in the art can also be used. Additionally, we have described a bolt or screw mechanism that reaches from the dorsal handle member to the ventral handle member entering the latter from the side after making a right angle turn. It should readily be understood by one skilled in the art that a direct connection can be effected where the screw or bolt passes through a single integrally formed bolt hole on each of the handles without making a right angle turn.

FIG. 3 to which reference is now made shows an isometric view of the ventral member 114 of the speculum shown in FIGS. 1A and 1B. The elements in the Figure are numbered as in FIGS. 1A and 1B but because the ventral member 114 is shown alone, identification of the elements are clearer. This is particularly true of hinge pins 16 and ventral member bolt hole 34.

FIGS. 4A and 4B show isometric and side views of insert support element 60 which is constructed to be positioned near the concave side of dorsal blade member 12. The concave and convex sides of dorsal blade member 12 and ventral blade member 14 are substantially as shown in the profile view of blade members 12 and 14 that appear in FIG. 2. In FIGS. 1A and 1B discussed above, insert support element 60 is shown with broken lines.

As shown in FIGS. 4A and 4B, insert support element 60 may be substantially saddle-shaped where fenders 66 extend from both sides of seat 62 substantially transversely. Protruding from the proximal portion of seat 62 is nipple 70. At the end of fenders 66 are engagement prongs 64 bounding fender notch 65 that can detachably engage with hinge pins 16 of ventral member 114. In some embodiments, holes of a diameter suitable for detachably engaging hinge pins 16 may be used instead of engagement prongs 64 and fender notch 65. Fenders 66 of insert support element 60 are positioned between ventral member 114 from which hinge pins 16 extend and dorsal member 112 in which hinge pin holes are positioned. Engagement prongs 64 and fender notch 65 are positioned so that hinge pins 16 pass through fender notch 65 and then enter the hinge pin holes in dorsal member 112.

Insert support element 60 is made from materials similar to the rest of the plastic speculum, for example polycarbonate, preferably transparent.

Insert support element 60 is not glued, welded or otherwise permanently attached to any portion of speculum 10.

Insert support element 60 may be used in addition to strengthening speculum 10 as a smoke remover. Nipple 70 is hollow and may be pneumatically connected to a suction source via tube 71, best seen in FIGS. 6A and 6B discussed below, so as to withdraw smoke and other residue resulting from electrosurgical or laser ablation gynecological procedures.

Typically, insert support element 60, including nipple 70, may be formed as an integral element using injection molding. In some embodiments, insert support element 60 may have a threaded male proximal end onto which a complementary threaded hollow elongated female element, nipple 70, may be screwed.

In order to further enhance smoke removal, apertures 68 may be constructed in seat 62 of insert support element 10.

The contour of seat 62 is such that it effectively forms a channel with the interior surface, that is, the concave surface, of dorsal blade member 12 as seen in FIG. 2 and discussed above. Smoke or debris resulting from an electrosurgical or laser ablation gynecological procedure may be drawn off by suction through such channel as shown in FIGS. 2 and 6A. In FIG. 6A, tube 71 is connected to a suction source; the source itself is not shown.

Seat 62 of insert support element 60 may be flatter or more curved than that shown in FIG. 4B. Both a flatter or more curved shape for insert support elements 60 permit effective channeling of smoke and/or detritus to nipple 70 and out of the speculum when a suction source is applied.

In FIG. 5, in another embodiment of the present invention, insert support element 60 is shown constructed with a retainer ring 75 through which an electrical connection of a light source, typically a light emitting diode (LED), and/or optical equipment may be positioned.

While not intended to be bound to a specific analysis of the forces involved, it is possible that the following analysis explains, at east partially, why the speculum of the present invention exhibits greater mechanical strength than conventional plastic specula and does not fracture at unacceptably low loads. FIG. 6A shows the forces believed to be acting on speculum 10 of the present invention. When inserted into the vagina during an examination, forces F_{T} act substantially perpendicular (vertical) to blades 12, 14 trying to return the blades from their open to their closed configuration. An upward force from the pins is generated and transferred via fenders 66 to the arcuate-shaped proximal region of seat 62. This arcuate-shaped region of seat 62 is positioned adjacent to the arcuate proximal end of dorsal blade member 12 the latter forming together with lateral walls 61 of dorsal handle 26, inspection aperture 63 as shown in FIGS. 6A-6B.

It is also believed that shear forces are better transferred and dispersed away from the hinge region by insert support element 60. FIG. 6B is a Figure of the speculum of the present invention showing two axes AA′ and BB′. Axis BB′ is situated above the pin region while AA′ is positioned below the pin region. FIG. 6C show cross sectional views along these axes. Below the pin region (AA′), a shear force F_{S} operates only on an area xy as insert element 60 does not extend below the pin region. Above the pin region where the insert element is located, shear force Fs operates on an area xy+xz, the area provided by the dorsal member lateral wall 61 having a thickness y and the insert element having a thickness z. Therefore at AA′, shear=Fs/xy while at BB′, shear=Fs/(xy+xz). The shear at AA′ being less at BB′ indicates that the shear stress has been transferred, and fracture around and above the hinge region is less likely.

It should be evident to one skilled in the art that while we have described speculum 10 of the present invention as having hinge pins 16 on the ventral member 114 and hinge holes on the dorsal member, the invention can be effected with a construction where the hinge pins are located on the dorsal member 112 and the hinge holes on the ventral member 114.

Plastic specula constructed according to the present invention have a destructive testing strength of at least about 6 kg, and therefore like metal specula they can be used in long duration gynecological procedures, including surgical procedures. Additionally, it is useful for gynecological out-patient procedures for which weaker specula are unsuited, such as colposcopy.

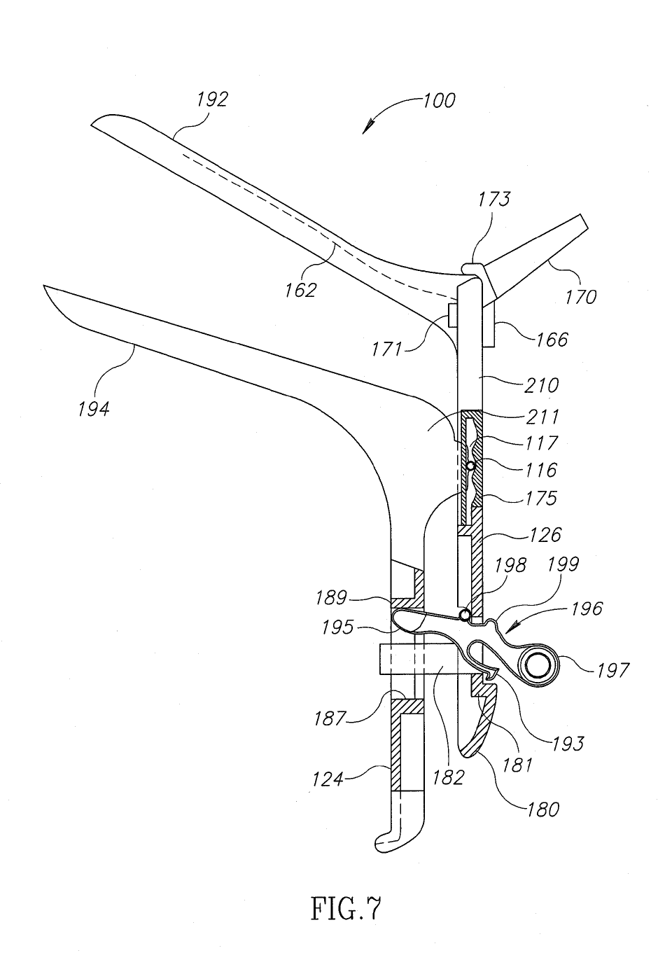

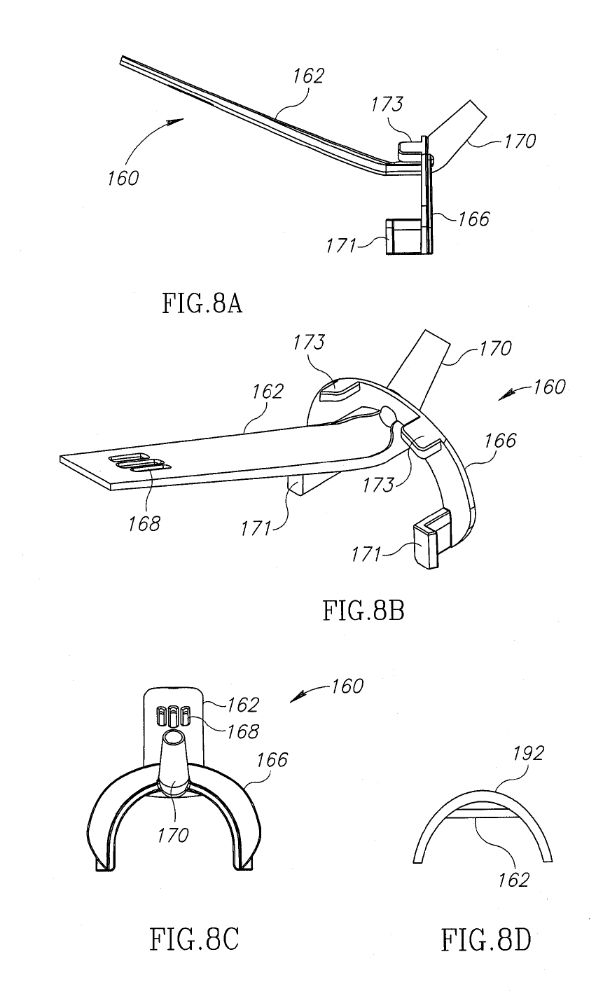

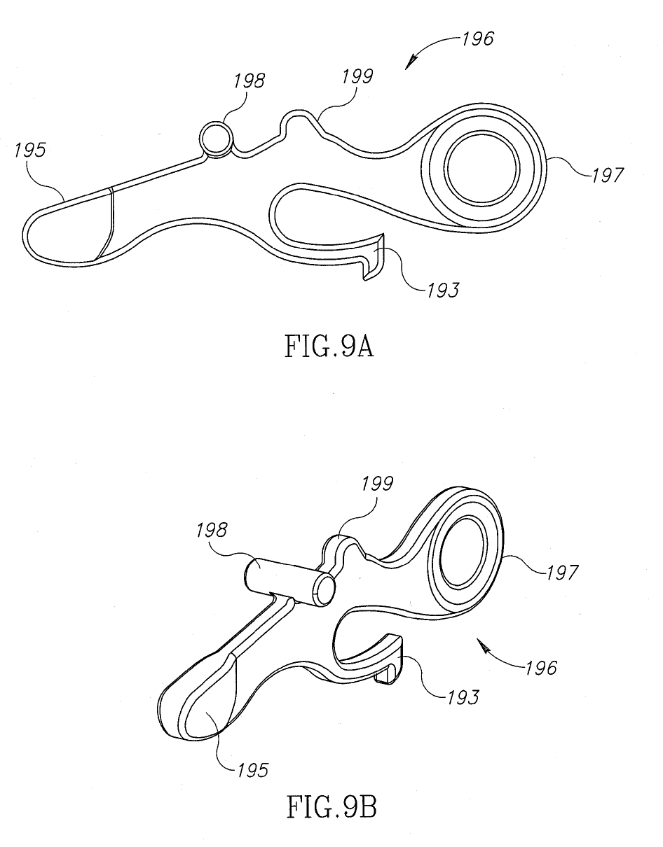

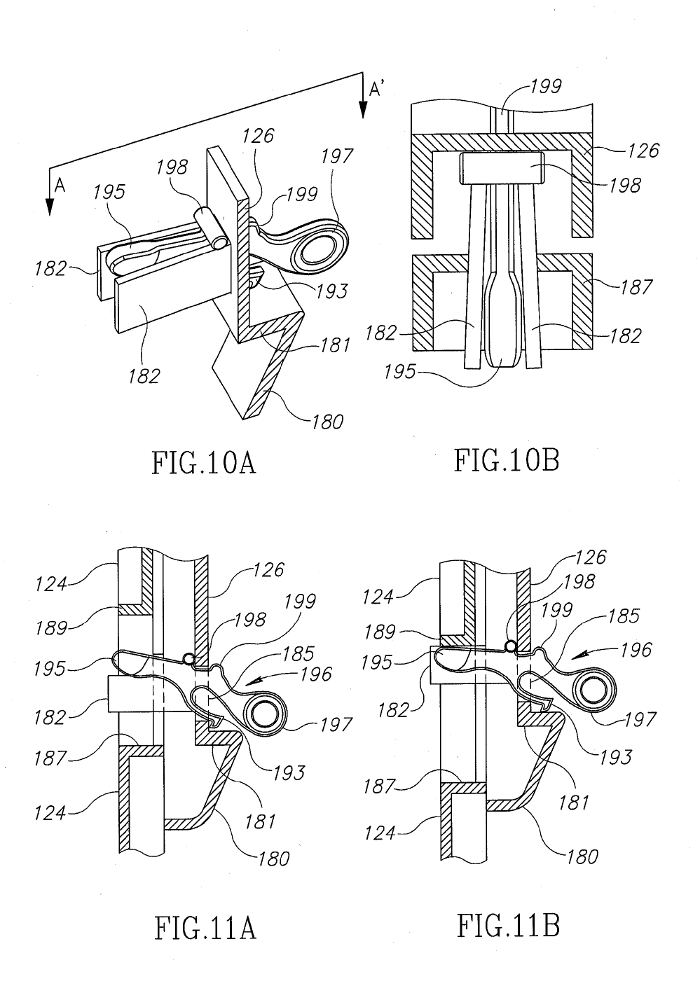

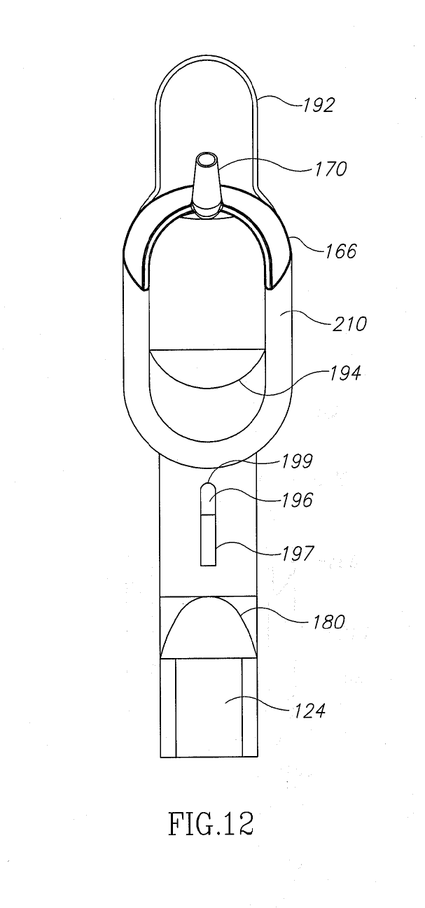

Reference is now made to FIGS. 7-12 in which another embodiment of a disposable speculum 100 and an insert support element 160 is shown. FIG. 7 is a side view of speculum 100 in its open configuration while FIGS. 8A-8C show side, isometric, and proximal views of an insert support element 160 usable with speculum 100 in FIG. 7. FIG. 8D shows the relationship between the dorsal blade 192 and the seat 162 of insert support element 160. FIGS. 9A and 9B show side and isometric views, respectively, of a lock element 196 used with speculum 100 shown in FIG. 7. FIGS. 10A and 10B present lock element 196 of FIGS. 9A and 9B positioned in speculum 100 of FIG. 7. FIGS. 11A and 11B show lock element 196 in its unlocked and locked position, respectively, while FIG. 12 is a proximal view of speculum 100 of FIG. 7.

Reference is now made to FIG. 7. Speculum 100 shown therein is formed of an injection molded plastic, in the present invention a high strength plastic, such as polycarbonate. Other resins or other materials possessing similar mechanical strength may also be used to form the specula of the present invention. In all cases, the plastic must be stronger and more durable than currently employed polystyrene or polypropylene. Typically, the plastic is a clear transparent plastic but colored or non-transparent high strength plastics may also be used

Speculum 100 is formed of a dorsal member and a ventral member. The dorsal member contains a dorsal blade member 192 and a dorsal handle 126, the latter typically integrally formed with blade member 192.

The ventral member includes a ventral blade member 194, a ventral handle 124 typically integrally formed with blade member 194, and a pair of hinge pins 116 (one being obscured), one hinge pin on each side of ventral blade member 194 near its proximal end 211 where it joins ventral handle 124. Hinge pins 116 can also be formed as studs, trunnions and the like, and, typically, are integrally formed with the ventral member.

Hinge pins 116 are insertable into hinge groves 117, one hinge groove 117 formed on each of a pair of lateral wall elements 210, thereby engaging dorsal and ventral members so that speculum 100 is ready for use. Lateral wall elements 210 connect between and are typically integrally formed with dorsal blade member 192 and dorsal handle 126. Hinge pins 116 in hinge groove 117 form a hinge around which dorsal blade member 192 pivots when speculum 100 is brought from its closed to its open configuration or vice-versa. Closed and open configurations have the same meaning in this embodiment as they have in previously described embodiments. In the closed configuration, dorsal blade member 192 lies facing and substantially adjacent to ventral blade member 194 essentially as shown in FIGS. 1B and 2. FIG. 7 represents the open configuration of speculum 100.

The inside surface of lateral wall elements 210 is the surface that partially defines the inspection aperture of the speculum used by a physician during examinations. The proximal arcuate surface of dorsal blade 192 defines the remainder of the inspection aperture.

In FIG. 7, a seat 162 of an insert support element 160, discussed in greater detail below in conjunction with FIGS. 8A-8B, is shown by broken lines adjacent to dorsal blade element 192.

The front profile of blade members 192 and 194 of speculum 100 discussed in conjunction with FIG. 7 is very similar to the profile presented in FIG. 2 when the blade members are in their closed configuration. Seat 62 of insert support element 60 in FIG. 2 is the equivalent of seat 162 of insert support element 160, discussed below with reference to FIGS. 8A-8C. As in FIG. 2, seat 162 and the concave side of dorsal blade member 192 shown in FIG. 8D form a channel through which smoke and other detritus produced during a gynecological procedure may be evacuated from speculum 100.

FIG. 12, to which reference is now made, is a proximal view, i.e. back view, of the speculum shown in FIG. 7.

FIGS. 8A-8C are side, isometric and proximal views of insert support element 160 which is constructed to be positionable adjacent to the concave side of dorsal blade member 192 as in FIG. 7. The concave and convex sides of dorsal blade member 192 and ventral blade member 194 are substantially the same as shown in the profile view of blade members 12 and 14 that appear in FIG. 2. FIG. 8D shows the relationship of the concave side of dorsal blade member 192 and seat 162 of insert support element 160.

As shown in FIGS. 8A-8C, insert support element 160 is substantially saddle-shaped with fenders 166 extending generally transversely from both sides of seat 162. Protruding from the proximal portion of seat 162 is nipple 170. At the end of fenders 166 are engagement flanges 171 that compressively engage with the arc-shaped surface formed at the junction of dorsal blade member 192 and lateral wall elements 210 of dorsal handle 126. Additionally, flange elements 173 compressively engage with the arc-shaped surface at the junction of dorsal blade member 192 and lateral wall elements 210. The arc-shaped surface discussed here is obscured by fenders 166 in FIGS. 7 and 12.

In other embodiments, rather than compressively attaching insert support element 160 with the dorsal member, receiving holes may be formed near the arc-shaped region discussed above to receive flanges 171 and 173, thereby effecting attachment.

Insert support element 160 is made from materials similar to the rest of the plastic speculum, for example polycarbonate. As would be evident to one skilled in the art, when support element 160 is compressively attached to the arc-shaped surface described above, a resilient material, such as polycarbonate, would be required.

In addition to strengthening speculum 100, insert support element 160 may be used as a smoke remover. Nipple 170 is hollow and may be pneumatically connected to a suction source (not shown) via a tube (also not shown), so as to withdraw smoke and other residue resulting from electrosurgical or laser ablation gynecological procedures.

Typically, insert support element 160, including nipple 170, may be formed as an integral element using injection molding. In some embodiments, insert support element 160 may have a threaded male proximal end onto which a complementary threaded hollow elongated female element, nipple 170, may be screwed.

In order to further enhance smoke removal, apertures 168 may be formed in seat 162 of insert support element 160.

The contour of seat 162 is such that it effectively forms a channel with the interior surface, that is, the concave surface, of dorsal blade member 192 as seen in FIGS. 7 and 8D discussed above.

Seat 162 of insert support element 160 may be substantially flat as shown in FIGS. 8A-8B or more curved than that shown there. Both a flat or curved shape for seat 162 of insert support element 160 permit effective channeling of smoke and/or detritus to nipple 170 and out of the speculum when suction is applied.

While locking mechanisms similar to those previously described with other embodiments may be used with the speculum in FIG. 7, another type of lock element may also be used. This lock element, its positioning in the speculum and its operation are shown in FIGS. 9A-9B, 10A-10B and 11A-11B respectively.

Side and isometric views of lock element 196 are shown in FIGS. 9A-9B, respectively, reference to which is now made. Lock element 196, also herein denoted as trigger element 196, is typically a unitary element with a wedge end 195, a press end 197, a pin structure 198 lying essentially transverse to the length of trigger element 196, a positioning protrusion 199 lying between press end 197 and pin structure 198, and a trigger leg 193. Trigger leg 193 extends from the ventral side of the body of trigger element 196 opposite to the side on which pin structure 198 and positioning protrusion 199 are located. The body of trigger element 196 is substantially of uniform thickness except for wedge end 195 which is thicker.

Without intending to be limiting, trigger element 196 is typically integrally molded from a plastic, such as nylon, polyethylene, polypropylene, acetal (polyoxymethylene) and polycarbonate.

FIG. 10A shows the positioning of trigger element 196 in an aperture 185 in dorsal handle 126. Aperture 185 through which trigger element 196 extends is obscured in both FIGS. 10A and 10B but is shown in FIGS. 11A and 11B. Aperture 185 is situated between pin structure 198 and positioning protrusion 199 when trigger element 196 is inserted therethrough. Pin structure 198 rides above a pair of substantially parallel positioning projections 182, the latter protruding essentially transversally from dorsal handle 126 in the distal direction. Since wedge end 195 of trigger element 196 is thicker than the remainder of element 196, when wedge end 195 is moved to its locked position it becomes wedged in between positioning projections 182. FIG. 1013 is a top view of the positioning of trigger element 196 between positioning projections 182 along axis AA′. FIG. 10A identifies the A-A′ axis. As shown in FIG. 10B, wedged wedge end 195 causes a slight deviation of positioning projections 182 from their initial generally parallel configuration.

In FIGS. 10A and 10B positioning projections 182 do not contain a ratchet mechanism. In other embodiments, projections 182 may contain a ratchet mechanism.

Turning to FIGS. 11A and 11B, to which reference is now made, the position of trigger element 196 is shown when blade members 192 and 194 and handles 124 and 126 are movable (FIG. 11A), i.e. when element 196 is in its unlocked position, and when the blade members and the handles are not movable (FIG. 11B), i.e. when element 196 is in its locked position. When handles 124 and 126 are movable with respect to each other, blade members 192 and 194 may be separated or brought close together by pivoting the blades around pin 116. Additionally, the blade members may be separated from each other by moving handles 124 and 126 so that pins 116 move along ratchet track 175 (FIG. 7). Thus, the present speculum 100 has two degrees of freedom, a rotation around pins 116 and a translation motion of pins 116 along track 175.

After speculum 100 is brought from its closed configuration, substantially the same configuration as in FIG. 1B, to its partially or completely open configuration as in FIG. 7, trigger element 196 is used to lock handles 124 and 126 so that blade members 192 and 194 are in the fixed position selected by the physician for carrying out the required gynecological procedures.

Locking of speculum 100 when the height of dorsal blade 192 vis-à-vis ventral blade 194 and/or the desired angle separating the blades has been reached, is effected by pushing press end 197 of trigger element 196 upward. Trigger element 196, situated in aperture 185 in the lower portion of dorsal handle 126 rotates so that wedge end 195 moves downward as in FIG. 11B in between positioning projections 182. Semi-flexible trigger leg 193 rests on the edge of ledge 181 of the lower portion 180 of dorsal handle 126. This locks the dorsal 126 and ventral 124 handles and prevents further rotation and/or translation of blade members 192 and 194. Trigger leg 193 acts as a spring pushing trigger element 196 up so that element 196 can move out of its locked position while retaining pin structure 198 as a rotational axis.

To unlock, i.e. open, locked speculum 100, the user pushes down on press end 197 of trigger element 196 causing wedge end 195 to rotate upward and out from between positioning projections 182.

It should readily be understood by one skilled in the art that an advantage of the present locking mechanism is that locking and unlocking of speculum 100 can be effected with one hand. This leaves the second hand free to perform other operations required by the gynecological procedures being executed.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. Therefore, it will be appreciated by persons skilled in the art that the present invention is not limited by what has been particularly shown and described herein above. Rather the scope of the invention is defined by the claims that follow.

1. A vaginal speculum comprising:
A. a plastic dorsal member having a distal end and a proximal end which includes:
i. a curved dorsal blade member with concave and convex surfaces,
ii. a dorsal handle, and
iii. a pair of lateral wall elements connecting between and formed integrally with said blade member and said handle so as to form an inspection aperture between said concave surface of said blade member and an opposing portion of said handle,
wherein each said wall element has formed therewith a first hinge portion;
B. a plastic ventral member which includes:
i. a curved ventral blade member,
ii. a ventral handle having a portion adapted to fit between said lateral wall elements of said dorsal member and having formed thereon a pair of second hinge portions adapted for interconnection with said first hinge portions, thereby to form a hinge between said dorsal member and said ventral member for facilitating relative angular rotation therebetween; and
C. an insert support element in mechanical connection with said proximal end of said dorsal blade member arranged within said inspection aperture and configured to extend within said concave surface of said dorsal blade member so as to cooperate therewith to form a channel for the evacuation of smoke or detritus produced by a gynecological procedure when suction from a suction source is applied thereacross, said support element in load-bearing arrangement with a predetermined one of said first or second pairs of hinge portions, thereby to reduce the likelihood of stress induced mechanical failure of said speculum when in use.

2. A vaginal speculum according claim 1 wherein the relative angular rotation is effected about an axis extending transversely through said hinge, adapted to support thereat shear force components normal to said axis above the hinge.

3. A vaginal speculum according to claim 1 wherein said insert support element comprises:
i. a seat having a proximal portion formed to fit within said concave surface of said dorsal blade element and a distal portion extending generally away from said inspection aperture; and
ii. a pair of lateral fenders extending generally transversely away from said seat along said pair of lateral wall elements so as to be in load-bearing arrangement with said hinge, thereby to reduce the likelihood of sheaf stress induced mechanical failure of said speculum when in use.

4. A vaginal speculum according to claim 3 wherein forces acting on said hinge are partially transferred by said fender elements to said arched proximal end of said dorsal blade member.

5. A vaginal speculum according to claim 3, said insert support element further comprising a hollow nipple adapted for connection to a suction source to evacuate smoke and/or detritus produced as a result of the gynecological procedure.

6. A vaginal speculum according to claim 3, said insert support element further comprising a retainer ring through which an electrical connection of a light source or an optical instrument may be positioned.

7. (canceled)

8. A vaginal speculum according to claim 1 wherein each of said first hinge portions includes a hinge pin and each of said second hinge portions includes a hinge hole.

9. A vaginal speculum according to claim 1 wherein each of said second hinge portions includes a hinge pin and each of said first hinge portions includes a hinge hole.

10. A vaginal speculum according to claim 9, wherein each of said lateral wall elements of said dorsal member includes a hinge pin lead slot leading to said hinge holes from the distal edge of said lateral wall elements along their inside surfaces.

11. A vaginal speculum according to claim 1 wherein said dorsal blade member and said dorsal handle member are integrally joined and form an arcuate region at said proximal end of said dorsal blade member

12. A vaginal speculum according to claim 1, wherein forces acting on said hinge portions are transferred by said fender elements to said arcuate region of said seat said arcuate region of said seat thereby at least partially supporting the arcuate region at said proximal end of said dorsal blade member

13. A vaginal speculum according to claim 1 wherein shear forces acting on said hinge portions are dispersed therefrom because of the greater area of the speculum upon which they act above the hinge region than below it, such greater area resulting from said fender elements being positioned essentially above the hinge region.

14. A vaginal speculum according to claim 1 wherein said second hinge portions are hinge pins and said first hinge portions are elongated grooves fitted with a ratchet mechanism into which said hinge pins are inserted.

15. A vaginal speculum according to claim 1 further comprising a locking mechanism in mechanical communication with said dorsal and ventral handle members operable to lock said dorsal blade member and said ventral blade member in a user-selected position with respect to each other.

16. A vaginal speculum according to claim 15, wherein said locking mechanism further comprises an at least partially threaded screw that is joined to and extends from said dorsal handle member through said ventral handle member and having a nut positioned thereon for holding said ventral and dorsal blade members in a predetermined user-selected position with respect to each other.

17. A vaginal speculum according to claim 15, wherein said locking mechanism is a trigger mechanism in mechanical communication with said dorsal and ventral handles operable to lock said handles so that said dorsal and ventral blade members remain in a user-selected position with respect to each other.

18. A vaginal speculum according to claim 17, wherein said trigger mechanism comprises a trigger element configured so that when pressed in a first direction said trigger element is operative to lock said ventral and dorsal handles thereby preventing rotation and/or translation of said blade members, and when pressed in a second direction said trigger element is operative to unlock said handles, thereby allowing rotation and/or translation of said blade members.

19. A vaginal speculum according to claim 17, wherein said trigger mechanism comprises:
a pair of substantially parallel positioning projections, said projections separated from each other by a predetermined distance and projecting generally transversely from said dorsal handle in a distal direction; and
a trigger element having a proximal press end and a distal wedge end, said wedge end being formed to be thicker than the remaining sections of the trigger element,
wherein when said press end is pressed in a first direction said wedge end is moved between said positioning projections and wedged therebetween locking said ventral and dorsal handles, thereby preventing rotation and/or translation of said blade members, and when said press end is pressed in a second direction said wedge end moves out of and away from said positioning projections unlocking said ventral and dorsal handles, thereby allowing rotation and/or translation of said blade members.

20. An insert support element for use with a vaginal speculum, said speculum configured to include an inspection aperture, hinge pins forming part of a hinge and a dorsal blade element having a concave surface, said support element comprising:
i. a seat having a proximal portion formed to fit within the concave surface of the dorsal blade member and having a distal portion extending generally away from the inspection aperture; and
ii. a pair of lateral fenders extending generally transversely away from said seat so as to be in load-bearing arrangement with the hinge of the speculum, thereby to reduce the likelihood of stress induced mechanical failure of the speculum when in use, and
wherein said distal portion and said proximal portion of said seat are configured so as to cooperate with the concave surface of the dorsal blade member of the speculum to form a channel for the evacuation of smoke or detritus produced by a gynecological procedure when suction from a suction source is applied thereacross.

21. A vaginal speculum comprising:
A. a plastic dorsal member having a distal end and a proximal end and which includes a dorsal blade member with concave and convex surfaces, a dorsal handle, and a pair of lateral wall elements connecting between and formed integrally with said blade member and said handle, and wherein each of said wall elements has formed therewith a first hinge portion;
B. a plastic ventral member which includes a ventral blade member and a ventral handle having a portion adapted to fit between said lateral wall elements of said dorsal member and having formed thereon a pair of second hinge portions adapted for interconnection with said first hinge portions thereby to form a hinge; and
C. an insert support element connected to said proximal end of said dorsal blade member and arranged and configured to extend within said concave surface of said dorsal blade member so as to cooperate therewith to form a channel for the evacuation of smoke or detritus when suction from a suction source is applied thereacross, said support element in load-bearing arrangement with a predetermined one of said first or second pairs of hinge portions, thereby to reduce the likelihood of mechanical failure of said speculum when in use.
